(19) United States
(12) Patent Application Publication
(10) Pub. No.: US 2022/0409354 A1
(43) Pub. Date: Dec. 29, 2022

(54) INTEGRATED RADIAL SILICONE-FILLED CELL-STRUCTURED HUMAN BODY IMPLANT AND MANUFACTURING METHOD THEREOF

(71) Applicant: Megen Limited, Seoul (KR)

(72) Inventor: Ki Young Youn, Guri-si (KR)

(21) Appl. No.: 17/514,478

(22) Filed: Oct. 29, 2021

(30) Foreign Application Priority Data
Jun. 28, 2021 (KR) 10-2021-0084011

(51) Int. Cl. A61F 2/00

(52) U.S. Cl. CPC A61F 2/0059; A61F 2250/0003; A61F 2230/0013; A61F 2210/0085; A61F 2240/001

(57) ABSTRACT

The present invention relates to an integrated radial silicone-filled cell-structured for human body implant and manufacturing method thereof, and more particularly, in a human body implant, to an integrated radial silicone-filled cell-structured human body implant comprising: a first silicone-filled cell including a silicone filling material, in which the silicone filling material is formed in the center of the implant; and a second silicone-filled cell surrounding an outer surface of the first silicone-filled cell, being formed radially around the center of the implant, and including a silicone filling material formed with a cross-linking density different from a silicone cross-linking density of the first silicone-filled cell.

### CROSS-REFERENCE TO RELATED APPLICATIONS

This application claims priority to Korean Patent Application No. 10-2021-0084011 filed on Jun. 28, 2021, the entire disclosure of which is expressly incorporated herein by reference.

### STATEMENT RE: FEDERALLY SPONSORED RESEARCH/DEVELOPMENT

Not Applicable

### BACKGROUND

### Technical Field

The present invention relates to an integrated radial silicone-filled cell-structured for human body implant and manufacturing method thereof, and more particularly, to an integrated radial silicone-filled cell-structured human body implant and manufacturing method thereof which blocks infection resulting from bleeding and blooming and improves biocompatibility by introducing an integrated radial silicone-filled cell structure in order to reduce the high incidence rate of capsular contracture, rupture, and anaplastic large-cell lymphoma of conventional human body implant.

### Background Art

The use of silicone implant has been restricted by the United State Food and Drug Administration (FDA) since 1992, but since 2006, the use of silicone implant filled with less cohesive gel was approved again, and from 2012 and 2013, the use of products filled with a highly cohesive gel has been approved and has been widely used in earnest so far.

Looking at the history of silicone implant, the first breast implant was developed by Cronin and Gerow in 1962 and produced and marketed by Dow Corning from 1964 to 1968. Since the development of the silicone implant, operators have experienced various complications, and the evolution of the implant has been repeated in an effort to overcome this.

The characteristic of the first generation of silicone implant used from 1962 to 1970 was that the shell was thick and the inside was composed of a high-viscosity gel, and it was a teardrop-shaped implant having a seam on the outside designed in a way that a piece of Dacron cloth was attached to the back surface to fix the implant in place as inserted. The problem with this implant is that the incidence rate of capsular contracture is high, and to overcome such capsular contracture, a new silicone implant began to appear in the mid-1970s.

The second generation of silicone implant, used from the 1970s to 1982, had a round shape in which the seams were removed, and the shell became thinner and consisted of a low-viscosity gel, thereby having a characteristic soft feel. There was no Dacron piece of cloth attached to the back side, and it had a smooth surface. However, this implant did not reduce the incidence rate of capsular contracture, but rather the thin shell was easily ruptured, thereby occurring bleeding in which the silicone gel seeped through the shell.

The third generation was used from the early 1980s to the early 1990s, in which efforts were made to reduce the phenomenon of seeping of silicone gel by strengthening the strength and durability of the shell. It consisted of a thick barrier coating with silica and a more viscous gel, thereby trying to increase the life of the shell and reduce spheroid contracture and silicone gel migration. Such implant characteristic was also observed in the 4th and 5th generations of implant.

The 4th generation has been used from 1993 to the present and is manufactured with 3rd generation technology and is a product containing cohesive silicone gel with increased viscosity. An additional feature is that a texturization technique was introduced to make the surface of the implant concave and convex considering that the incidence rate of capsular contracture is not reduced.

The 5th generation has been in use from 1993 to present and is harder than the 4th generation, and is a product containing cohesive silicone gel whose shape of implant is form-stable by external force. There are two types of products: smooth surface and concave-convex surface, and there are two types: anatomical shape and round shape. The most distinctive feature of the 5th generation is that the type of implant has become much harder and heavier than the previous generation.

The 6th to 8th generations are the heyday of cohesive gel implant, a period when gummy bear implant (gummy bear implants) take their place into a typical shape. The biggest feature of the gummy bear implant is that when the implant is cut into two pieces, the cohesive gel is not separated from the cut region but is attached while maintaining adequate elasticity. In addition, despite the rupture of the implant, the cohesive silicone gel did not bleed, so there was an expectation that secondary problems would not occur. Gummy bear implant has various product groups depending on the composition and characteristics of the inner and outer regions.

The implant filled with cohesive gel is a method of increasing the viscosity to prevent bleeding and blooming of the silicone contents compared to the past silicone implant, and is characterized by enhancing the property of maintaining the initial shape by maximizing cross-linking using a chemical cross-linking agent. As the degree of cross-linking increases, it feels like a soft cheese rather than a liquid, and an effect of reducing the leakage of the contents when the shell is damaged and seeping through the shell can be expected. The biggest feature of the 6th to 8th generations is that the degree of cross-linking of the cohesive gel is strengthened, and it became hard and thick at the same time as the surface layer was multi-layered in order to prevent wrinkling and shape deformation of the implant caused by the difference in softness with the surface layer. The characteristics of implant in this period are that unlike the expectation the incidence rate of capsular contracture was not decreased significantly, and that a new type of breast implant illness (BII) was emerged.

Breast augmentation implant has been changed in the direction to prevent capsular contracture and silicone gel bleeding that occurred in the early days of application, but considering the United State FDA report on the safety of breast implant filled with silicone gel, it was confirmed that the development of a new concept of innovative breast augmentation implant is urgently required.

According to the United State FDA report in 2011, it was confirmed that the frequency of removal surgery due to capsular contracture, rupture, asymmetry of the implant including these, large and small wrinkles, and malposition was remarkably high. In addition, the United State FDA report in 2019 drew attention as the frequency of suffering from breast implant illness symptoms increased dramatically compared to 2011. Most of the symptoms after breast implant are chronic fatigue and pain, insomnia, hair loss, memory loss, depression, gastrointestinal disorder, rash, dyspnea, myalgia, and joint pain that are difficult to explain medically. It is reported that these symptoms abruptly occurred in the case of using the 6th to 8th generation breast augmentation implant.

As a prior art, Korean Patent Registration No. 1067475 (Breast implant that has surface with silicone open cell foam layer, and its manufacturing method) discloses a technology that minimizes the occurrence of capsular contracture, a side effect that may occur after insertion of the implant into the body, by forming a silicone open cell foam layer on the shell surface of the implant, however, it does not disclose or imply for the problems caused by the implant shell structure and weight load.

Meanwhile, it also shows that breast implant associated-anaplastic large-cell lymphoma (BIA-ALCL), which was reported by the FDA in July 2019, is approaching a serious situation. At the time of the report, approximately 573 people were diagnosed with BIA-ALCL, and it was found that 481 of them used Allergan texture type implant. And 12 of the 33 deaths were found to be related to the company's products. In addition, as of October 2019, 809 people worldwide were confirmed as BIA-ALCL cases, and the global recall of Allergan's biocell breast implants and tissue expanders was taken, which caused a huge sensation.

Accordingly, the present inventors identified the cause of capsular contracture, rupture, anaplastic large cell lymphoma, and human body implant illness symptoms while trying to find a silicone implant that is effective in minimizing the human body implant illness symptoms while reducing the incidence rate of capsular contracture and anaplastic large cell lymphoma by maximizing compatibility with the body, while minimizing the various loads caused by the action and reaction due to the biomechanical interaction of the human body implant with the human body, and as a solution to this problem, the present invention was completed by developing an integrated radial silicone-filled cell-structured human body implant and manufacturing method thereof.

### BRIEF SUMMARY

### Technical Subject

The present invention is to solve the problems of the prior art, and an object of the present invention is to provide an integrated radial silicone-filled cell-structured human body implant and manufacturing method thereof capable of preventing the bleeding or blooming of the implant contents through the deformed region of the shell of a conventional human body implant.

Another object of the present invention is to provide an integrated radial silicone-filled cell-structured human body implant and manufacturing method thereof capable of reducing the concentration of loads over a long period of time on the implant body and interface due to the multi-layered structure and the seam between the patch and the injection valve of a conventional human body implant.

Yet another object of the present invention is to provide an integrated radial silicone-filled cell-structured human body implant and manufacturing method thereof capable of reducing the impact (load) affecting the human body by reducing the amount of silica contained in the human body implant.

Still another object of the present invention is to provide an integrated radial silicone-filled cell-structured human body implant and manufacturing method thereof, in which the implant composition is homogenized by optimizing the implant silicon composition and maximizing the degree of cross-linking in order to prevent any bleeding and blooming of the contents even if the human body implant is damaged.

Yet still another object of the present invention is to provide an integrated radial silicone-filled cell-structured human body implant and manufacturing method thereof capable of minimizing the strong stress and pressure applied to the human tissue due to the high strength and thickness of the conventional human body implant.

### Technical Solution

In order to achieve the above objects, the present invention provides, in a human body implant, an integrated radial silicone-filled cell-structured human body implant comprising: a first silicone-filled cell including a silicone filling material, in which the silicone filling material is formed in the center of the implant; and a second silicone-filled cell surrounding an outer surface of the first silicone-filled cell, being formed radially around the center of the implant, and including a silicone filling material formed with a cross-linking density different from a silicone cross-linking density of the first silicone-filled cell.

The integrated radial silicone-filled cell-structured human body implant according to according to an embodiment of the present invention, is characterized in that the human body implant is used for at least one selected from a group consisting of chest, breast, buttocks, nose, chin, forehead, calf, thigh, and wrinkles.

The integrated radial silicone-filled cell-structured human body implant according to according to an embodiment of the present invention, is characterized in that the human body implant has a semi-circular shape structure, wherein the first silicon-filled cell is formed in a semi-circular shape in the center of a lower surface of the implant of the semi-circular shape, and wherein the second silicon-filled cell surrounds an outer surface of the first silicon-filled cell in a semicircular shape and is radially formed around the center of the implant.

The integrated radial silicone-filled cell-structured human body implant according to according to an embodiment of the present invention, is characterized in that the degree of cross-linking of the human body implant is 95% or more.

The integrated radial silicone-filled cell-structured human body implant according to according to an embodiment of the present invention, is characterized in that the silicone filling material includes a silicone resin, a cross-linking agent, a catalyst, and a silica, wherein the total content of silica contained in the implant is between 0.1 vol % to 40.0 vol % in a temperature range of −20° C. to 40° C. based on the volume of the implant.

The integrated radial silicone-filled cell-structured human body implant according to according to an embodiment of the present invention, is characterized by comprising a plurality of silicone-filled cells disposed sequentially on an outer surface of the second silicone-filled cell.

The integrated radial silicone-filled cell-structured human body implant according to according to an embodiment of the present invention, is characterized in that the first silicon-filled cell has a cell density of 100% of a circular closed cell and a small-sized geometric cell structure, wherein the second silicon-filled cell has an open cell ratio close to an ellipse of a cell density between 30% and 70% and a geometrical cell structure whose size is relatively larger than that of the central region, and wherein the silicon-filled cells formed on the outermost surfaces of the plurality of silicon-filled cells has a cell density of about 90% of a long elliptical closed cell and a geometrical cell structure having the largest size.

In addition, the present invention, in a manufacturing method of a human body implant, provides a manufacturing method of an integrated radial silicone-filled cell-structured human body implant characterized by comprising the steps of: preparing a plurality of silicone solution mixtures having different degrees of cross-linking (S10); sequentially introducing the plurality of silicon solution mixtures into a mold (S20); and forming a plurality of radial silicon-filled cell structures by introducing a heated gas into a mixture of a plurality of silicone solutions having the different degrees of cross-linking (S30).

The manufacturing method of an integrated radial silicone-filled cell-structured human body implant according to an embodiment of the present invention may further includes the steps of: heating the plurality of radial silicon-filled cells to a temperature range of 70° C. to 100° C. at the same time as discharging the introduced input gas (S40); and obtaining a human body implant by cooling the heated plurality of silicon-filled cells to room temperature at a constant rate (S50).

The manufacturing method of an integrated radial silicone-filled cell-structured human body implant according to an embodiment of the present invention is characterized in that in the step S10, the plurality of silicone solution mixtures having different degrees of cross-linking has a degree of cross-linking of 40% or less, characterized in that in the step S30, a plurality of radial silicone-filled cells formed by injecting the heated gas into a plurality of silicone solution mixtures having different degrees of cross-linking has a cross-linking degree of 70% or less, and characterized in that in the step S40, the heated plurality of silicon-filled cells has a degree of cross-linking of 95% or more.

The manufacturing method of an integrated radial silicone-filled cell-structured human body implant according to an embodiment of the present invention is characterized in that the step S10 includes the step of passing the prepared mixture of the plurality of silicone solutions through a static stirrer having a heating device and a gas injection device.

The manufacturing method of an integrated radial silicone-filled cell-structured human body implant according to an embodiment of the present invention is characterized in that in the step S30, the mold has the shape of a implant corresponding to a human body to be implanted, wherein the heated gas is the gas injected through the lower hole of the mold and being heated by a heating device installed on an outer surface of the mold.

The manufacturing method of an integrated radial silicone-filled cell-structured human body implant according to an embodiment of the present invention is characterized in that the implant is composed of an upper portion and a lower portion.

The manufacturing method of an integrated radial silicone-filled cell-structured human body implant according to an embodiment of the present invention is characterized in that the human body implant is used for at least one selected from a group consisting of chest, breast, buttocks, nose, chin, forehead, calf, thigh, and wrinkles.

### Advantageous Effects

The manufacturing method of an integrated radial silicone-filled cell-structured for human body implant and manufacturing method thereof according the present invention is excellent in preventing or minimizing the formation of capsular contractures, the occurrence of ruptures, and the incidence of anaplastic large cell lymphoma, and the human body implant illness symptoms, which are commonly observed in conventional silicone and saline-based implants by blocking the phenomenon of bleeding or blooming of the implant contents through the deformed region of the shell of a conventional implant for injection into the human body.

In addition, the manufacturing method of an integrated radial silicone-filled cell-structured for human body implant and manufacturing method thereof according the present invention can reduce the formation of capsular contractures, the occurrence of ruptures, and the incidence of anaplastic large cell lymphoma by maximizing compatibility or affinity with the living body through application of an integrated implant in terms of structure and composition.

In addition, the manufacturing method of an integrated radial silicone-filled cell-structured for human body implant and manufacturing method thereof according the present invention minimizes the mechanical interaction between the implant and various tissues of the human body by reducing the amount of silica and dramatically lowering the weight and strength by applying a radial cell-structured implant, and may reduce the incidence of the human body implant illness symptoms by completely absorbing the impact and load inevitably applied to the implant region.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic diagram showing the structure of a breast tissue structure and a capsular contracture formation process around a breast implant.

FIG. 2 is a schematic diagram showing an immune response of a human body to foreign substances that are biodegradable.

FIG. 3 is a schematic diagram showing an immune response of a human body to a polymer material that is not biodegradable.

FIG. 4a is a graph showing changes in the characteristics of a scar tissue and a capsular contracture tissue according to the Baker stage.

FIG. 4b is a result of analysis of the photograph and thickness of the capsular contracture tissue.

FIG. 5 is a schematic diagram showing the difference in cross-linking of an epoxy resin and a silicone resin.

FIG. 6 is a graph showing changes in physical properties of an outer layer of a breast implant for each implant duration.

FIG. 7 is a schematic diagram illustrating a manufacturing process of a silicone breast implant.

FIG. 8 is an SEM photograph of the surface residues of a silicone breast implant.

FIG. 9 is a schematic diagram illustrating the phagocytosis of macrophages by size of foreign substances that are not biodegradable.

FIG. 10 is a schematic diagram showing the structural specificity of amino acids, which are components of collagen.

FIG. 11 is a result of SEM and adhesive strength analysis on the macro and micro texture characteristics of the surface of a silicon implant.

FIG. 12 is a schematic diagram illustrating an integrated implant in terms of structural and compositional aspects presented in the present invention.

FIG. 13 is a schematic diagram showing the effect of autoimmune disease and the reactivity of the silicone material on the symptoms of breast implant illness.

FIG. 14 is a schematic diagram showing mechanical properties of various tissues present in the chest region of the human body and loads in the chest tissues caused by various human activities.

FIG. 15 is a schematic diagram illustrating the biomechanical interaction between a chest implant and a chest region tissue.

FIG. 16 is a TGA analysis result for the weight loss of the silicone resin.

FIG. 17 is a schematic diagram illustrating an implant having a radial cell structure proposed in the present invention.

FIG. 18 is a schematic diagram illustrating the characteristics of a radial cell structure presented in the present invention.

FIG. 19 is a schematic diagram for explaining the manufacturing process of a breast implant of an integrated radial cell structure presented in the present invention.

FIG. 20 is a schematic diagram for explaining a curing peak analysis using a thermal analyzer according to an embodiment of the present invention.

FIG. 21 is a schematic diagram illustrating a curing peak analysis according to a dynamic heating test method using a thermal analyzer according to an embodiment of the present invention.

### DETAILED DESCRIPTION

Since the present invention can apply various transformations and can have various embodiments, preferred embodiments will be described in detail. It will be apparent to those skilled in the art that the present invention may be embodied in other specific forms without departing from the spirit and essential characteristics of the present invention. Unless otherwise defined, all technical and chemical terms and experimental methods used herein have the same meaning as commonly understood by a person skilled in the art to which the present invention belongs.

The present invention relates to an integrated radial silicone-filled cell-structured human body implant and manufacturing method thereof. By introducing an integrated radial silicon-filled cell structure in order to dramatically lower the high incidence rate of capsular contracture, rupture and anaplastic large-cell lymphoma of conventional human body implants, the present invention maximizes compatibility with the living body by fundamentally blocking the damage to the implant caused by infection and physiological side effects resulting from bleeding and blooming of the existing saline, silicone sol, silicone gel, or cohesive gel into the human body.

An integrated radial silicone-filled cell-structured human body implant according to an embodiment of the present invention includes a first silicone-filled cell and a second silicone-filled cell.

The first silicon-filled cell includes a silicone filling material, and the silicone filling material is formed in the center of a molded implant for human injection.

The second silicon-filled cell includes a silicone filling material that surrounds the outer surface of the first silicon-filled cell and is radially formed with respect to the center of the implant, and is formed with a cross-linking density different from that of the first silicon-filled cell.

In an integrated radial silicone-filled cell according to an embodiment of the present invention, a plurality of silicon-filled cells may be further formed on the outer circumferential surface of the second silicon-filled cell if necessary.

The conventional implant has a structural defect in which bleeding and blooming occur due to long-term concentration of stress and load applied between the implant and breast tissue around the seam and the multi-layered structure region. Especially, in the case of a seam containing an adhesive component as a main component, the possibility of bleeding increases due to a decrease in adhesive strength as a long period of time elapses.

On the other hand, the integrated implant according to the present invention does not have seams, patches, and multilayer structures, and has uniformity in terms of composition, and therefore it is possible to minimize the unreacted silicone resin and the unreacted silicone-based cross-linking agent caused by the incompleteness of the degree of cross-linking of the inner and outer layers of the conventional implant, and it is possible to minimize the instability of the dimensional stability associated with the free volume.

As such, the present invention can reduce the incidence of capsular contracture formation, rupture and anaplastic large-cell lymphoma by maximizing compatibility or affinity with the living body by applying an integrated implant in terms of structural and compositional aspects.

In addition, the present invention minimizes the mechanical interaction between the implant and various tissues of the human body by lowering the weight and strength by applying a radial cell-structured implant, and has the advantage of being able to lower the incidence of the human body implant illness symptoms by completely absorbing and dissipating the impact and load inevitably applied to the implant region.

The implant for injection into the human body according to the present invention can be used for at least one selected from a group consisting of chest, breast, buttocks, nose, chin, forehead, calf, thigh, and wrinkles, and is not limited thereto as long as it is a human body implant.

Hereinafter, as an embodiment of the present invention, a breast implant used for breast or breast among human tissues will be described in detail. However, these detailed descriptions should not be construed as being limited to human tissue and should be considered as exemplary.

The present inventors have identified that the resolution of capsular contracture and rupture and anaplastic large cell lymphoma is caused by the silicone composition applied to the implant, the degree of cross-linking, and the specificity of the implant manufacturing process, and this was achieved through the development of an integrated implant in terms of the structure and composition of the implant that fundamentally blocks it.

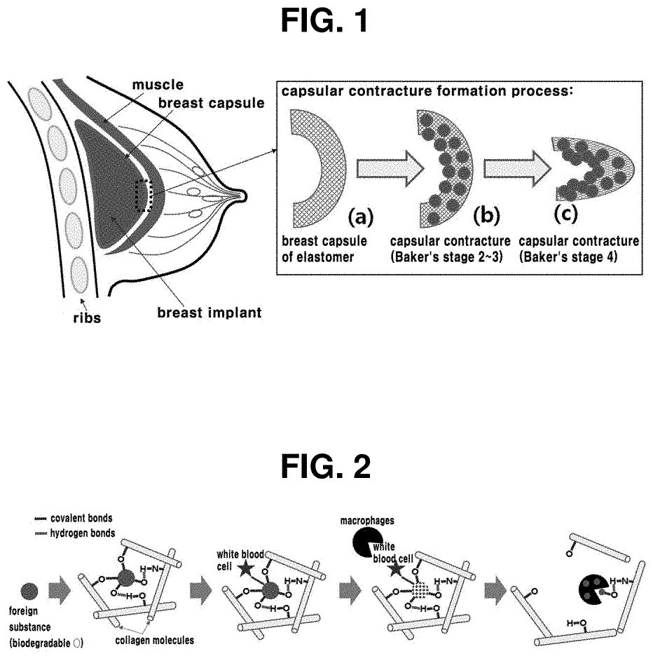

The origin of capsular contracture is derived from the breast capsule formed around the implant after surgery to insert a breast implant into the human body, and this breast capsule is also called scar tissue (hereinafter referred to as a scar tissue). Scar tissue is a kind of by-product that is naturally formed in the healing process, which is part of the immune response of the human body when surgery is performed or a wound is formed on the human body, and is composed of a complex of collagen of an elastomer, fibroblasts, and blood vessels. The scar tissue functions to place or support the breast implant in place, and also serves as a barrier to prevent or delay the diffusion of components inside the breast implant to the surrounding human tissue (FIG. 1).

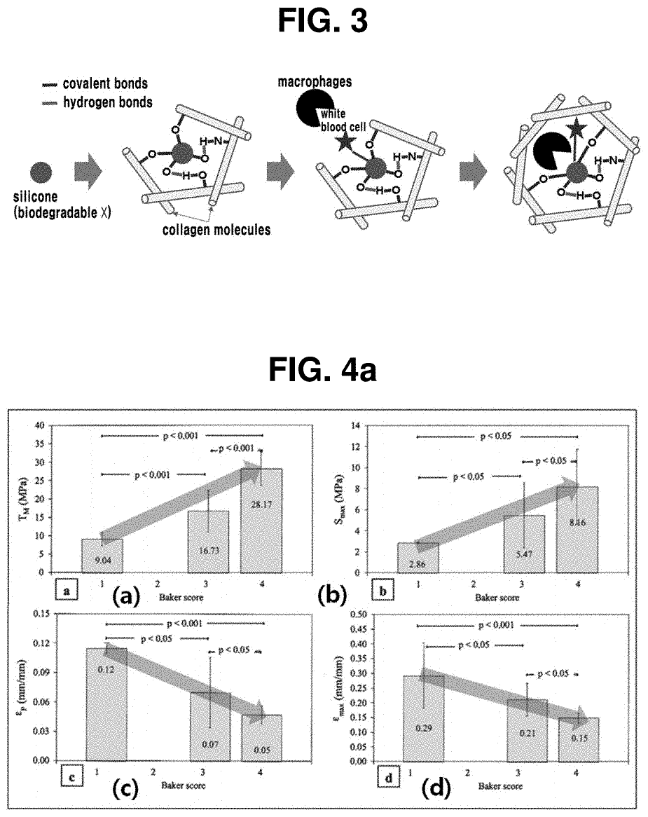

When foreign substances such as microorganisms infiltrate into the body, white blood cells break down foreign substances with the help of collagen (protein), and it is excreted out of the body through intracellular uptake, degradation and metabolism through phagocytosis of macrophages (FIG. 2). However, in the case of a silicone material that is not biodegradable, it is not degraded by the phagocytic activity of proteins including leukocytes and macrophages, so it has a limitation that it cannot be removed (FIG. 3).

Referring to FIG. 1, the film formed around the breast implant is composed of a complex of protein fibers of an elastomer. In general, after the immune response is completed, the high-viscosity collagen (protein) fiber complex is diluted with water in the body and separated and dispersed into individual protein fibers, so that scar tissue disappears. However, in the case of a silicone material, it has a property that it is not decomposed even by the in vivo immune response and phagocytosis of macrophages. In addition, in order to decompose the silicone material, more collagen molecules flock to form a complex of macrophages and leukocytes, and the like (FIG. 3). At this time, macrophages mobilize various types of catalysts present in the body as part of phagocytosis or secrete themselves to decompose silicone materials that cannot be decomposed. Among them, active oxygen in particular affects collagen molecules including silicone materials, and hydrogen and covalent bonds can be converted into a cross-linked structure after a long time elapses, so that an elastomer [FIG. 1(a)] is changed into a hard solid shape [FIG. 1(c)].

The rigid solid shape [FIG. 1(c)], due to the bleeding of a series of silicone materials and the continuously repeated immune action of the human body, and when the particle-form silicone material and collagen complex formed on the surface of the scar tissue elapses for a long time, the breast film of an elastomer made up of hydrogen bonds and covalent bonds, is transformed into a tissue of capsular contracture consisting of a rigid cross-linked structure. At this time, the capsular contracture tissue is crushed and the surface has a concave-convex shape, and due to this, the body's biomechanical activity puts pressure on the breast implant, thereby resulting in accelerated bleeding and blooming of the contents according to the dimensional deformation of the implant into the body. If this phenomenon continues for a long time, structural defects are generated by the load continuously applied to the surface of the implant, and cracks are generated through these regions, which eventually leads to rupture of the implant.

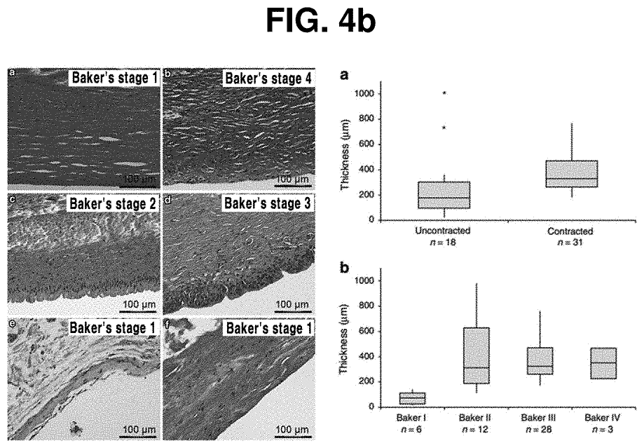

Evidence for the conversion of the aforementioned scar tissue of an elastomer into hard capsular contracture tissue can be found in numerous studies in academia. FIG. 4a is the research result on the correlation on the change in physical properties between the scar tissue and the capsular contracture tissue by Baker stage. Baker's stage 1 is a scar tissue of an elastic body, and Baker stage 4 is a tissue of capsular contracture. According to FIG. 4a, looking at the case of measuring the elastic modulus (a) and measuring the tensile strength (b), it can be confirmed that the tissue in which the capsular contracture occurs is harder than the scar tissue, and it was confirmed that the same trend was also observed in the results for strain, which is an index factor of elongation rate properties. In addition, according to FIG. 4b, when observing Baker's stage 4 in which tissue is capsular contracture, it has a fairly uneven surface different from the smooth surface of general scar tissue, and it was also confirmed that there was a significant difference in thickness compared to the case in which the capsular contracture was not performed in the Baker's stage 1.

Referring to FIGS. 4a and 4b, it was confirmed that the capsular contracture tissue is relatively hard compared to the general scar tissue and has severe surface irregularities, and if this capsular contracture tissue continuously applies a load to the implant, bleeding and blooming will occur, and it can be seen that if the surface irregularities are more severe, implant rupture may occur.

Bleeding and blooming of a breast implant filled with silicone gel has a direct correlation with capsular contracture, rupture, and anaplastic large-cell lymphoma, which is to be addressed technically in the present invention, and has an indirect correlation with breast implant illness symptoms.

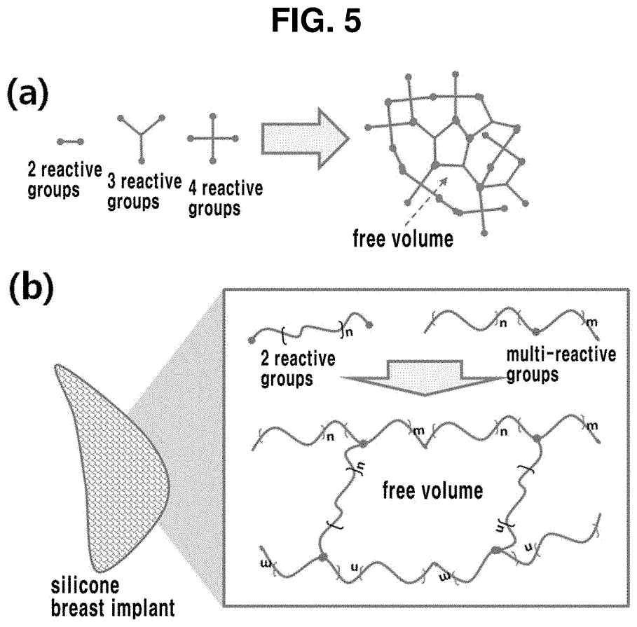

The cause of bleeding and blooming of existing silicone breast implants is also due to the structural specificity and degree of cross-linking of the silicone material. Referring to FIG. 5, the epoxy resin of (a) is formed in a cross-linked structure among 2 di-functional group usually small in molecular weight and having 2 reactive groups, tri-functional group with three reactive groups, and tetra-functional group with 4 reactive groups, and thus, has characteristics in which the free volume is very narrow and the strength of the cross-linked structure is strong. In addition, the cross-linking reaction of the epoxy resin has a small molecular weight and a large number of reactive groups, so that the reactivity proceeds quickly, and the degree of cross-linking becomes 95% or more.

On the other hand, silicone resin is composed of a main resin and a cross-linking agent. The main resin is a two-component liquid silicone rubber (LSR), and the cross-linking agent is a bi-functional group with two reactive groups, and it is preferable that the compound (siloxanes and silicones, dimethyl, vinyl terminated) have a relatively large molecular weight compared to the epoxy resin, but is not limited thereto.

Although a large amount of reactive groups of the main resin exist, since the molecular weight of the main resin is very large, reactive groups exist intermittently in the middle of the molecular structure of the main resin. For this reason, the cross-linked structure between them has a characteristic in which the free volume is quite wide and the strength of the cross-linked structure is weak as shown in FIG. 5(b). In addition, in the case of silicone resin, there are disadvantages in that because the molecular weight is very large and there are not many reactive groups, the molecular weight increases rapidly at the beginning of cross-linking, and the movement of macromolecules is rapidly reduced, and the reactivity proceeds very slowly, thereby limiting the degree of cross-linking to about 80%. In addition, the silicone cross-linked structure has very good elasticity, so when a specific external force is applied to the silicone-based implant, the cross-linked structure easily changes its dimensions and the free volume expands to a considerable level, and through this, the bleeding and blooming phenomena of the silicone material are accelerated. In addition, the low degree of cross-linking of the silicone resin means that there is a cross-linking agent or main resin that does not participate in cross-linking and moves separately. These unreacted cross-linking agents and main resins are substances that cause bleeding or blooming, and are a key cause of capsular contracture formation, rupture, and incidence of anaplastic large-cell lymphoma, which are to be technically solved in the present invention.

In particular, a breast implant with severe blooming phenomenon causes implant rupture by the load continuously applied by the capsular contracture tissue. The blooming phenomenon is a phenomenon in which an organic material having a rather large molecular weight is embedded in a gap of a free volume. Blooming is caused by interactions such as entanglement between free-volume structures and organic substances with high molecular weight, and as the expansion and aging of the free volume progress over a long period of time, the molecular structure gradually weakens, in particular, the polymer chain is broken in the defective region, thereby providing a cause for rupture. For example, when a rubber band is pulled by a certain force and elapsed for a long time in a taut state, microscopic deformation in the elastic band microstructure proceeds in the defect region due to the creep phenomenon, which is an inherent characteristic of polymers, and as a result, the rubber band breaks.

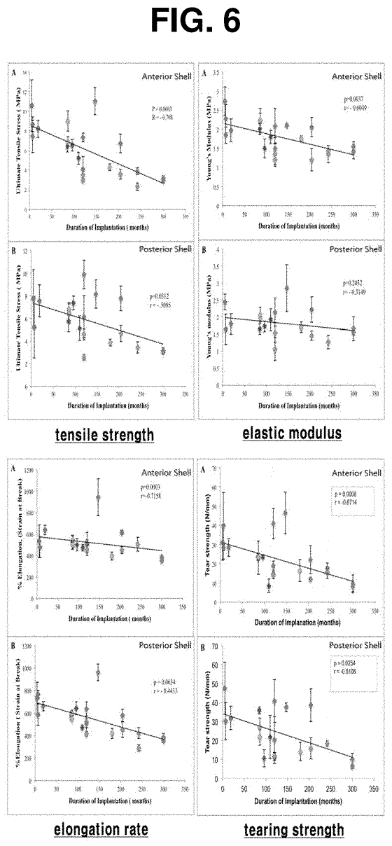

The possibility of implant rupture due to blooming was clearly confirmed through the results of numerous studies reported in academia. FIG. 6 is a result of measurement of the change in physical properties versus continual implant duration about the outer layer of the breast implant. Referring to FIG. 6, it can be seen that the tensile strength, the elastic modulus and the tearing strength are remarkably weakened with the continual progress after implant surgery, in particular, it was confirmed that the presence of the blooming phenomenon shows a phenomenon that the elongation rate characteristic is remarkably deteriorated as the duration of the implant progresses. Decreased elongation rate means that the silicone polymer chain is maximally stretched due to organic material having a large molecular weight caught in the free volume space of the cross-linked region of an elastomer, and it is evidence that the blooming phenomenon has occurred. In addition, the decrease in elongation rate due to blooming also acts as a cause of a decrease in tensile strength, elastic modulus, and tear strength. Therefore, when the decrease in elongation rate and strength reaches a certain critical point, it enters the stage of rupture, and it could be clearly predicted that there may also be a close relationship with anaplastic large-cell lymphoma, which is associated with the result of human immune action by organic matter that bleeds in large amounts due to rupture.

The root cause of the incidence of anaplastic large-cell lymphoma, including capsular contracture and rupture, is a phenomenon occurred by the complex entanglement of the above-mentioned bleeding or blooming phenomenon and the result of the body's immune action by organic matter that comes out through the bleeding and blooming.

In addition, it was confirmed that the specificity of the implant manufacturing process may also act as a key factor.

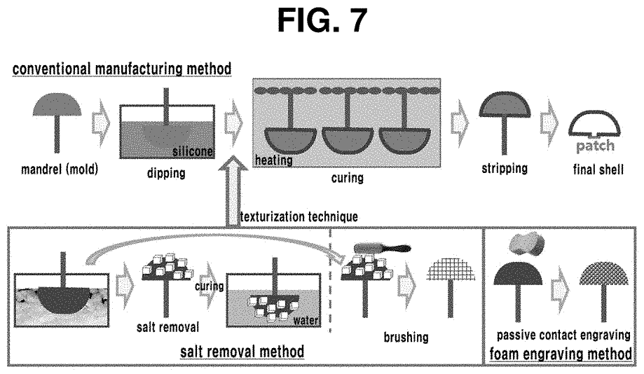

In the traditional silicon breast implant manufacturing process, as shown in FIG. 7, the overall process is performed manually. Briefly, a breast implant made of silicon with a smooth surface is first coated with a certain amount of silicone resin through a dipping process in a compounding machine containing high-viscosity liquid silicone, then the implant-shaped mandrel is hardened in the curing process and cooled, then removed from the mandrel, and then the coating is peeled off to obtain the implant shell. The final implant is completed after putting saline, silicone sol, silicone gel, or cohesive gel inside the shell. And as shown in the figure, there are two types of silicone implant manufacturing process with a concave-convex surface: salt removal method and foam engraving method. In the salt removal method, a silicone solution is coated on the mandrel, the salt particles are buried, and after curing and cooling, the salt is dissolved in water or removed with a brush to give the implant shell surface a concave and convex shape. In the foam engraving method, a silicone solution is applied to the mandrel and then rubbed or lightly contacted so that the implant shell is not deeply cut using a rubber or polyurethane foam product, and then peeled off to give a concave-convex shape.

The above-mentioned conventional manufacturing method is currently adopted by most companies that manufacture silicone breast implants. As described above, various problems are exposed due to the limitations of the conventional manual method. Looking at this roughly, first, the thickness of the implant shell may not be uniform. The thickness of the implant is controlled by preheating the mandrel or lowering the viscosity by controlling the temperature of the mixing bath containing the high-viscosity silicone solution. The problem with this process is that the silicone solution is a reactive mixture, and as time elapses with an applied temperature condition, a slight cross-linking reaction proceeds, resulting in a gradual rise in viscosity. Therefore, manufacturing an implant shell having a certain coating thickness is fundamentally a cause of technical problems. As the non-uniform coating thickness passes through the curing process, a difference in the degree of cross-linking occurs due to the difference in thermal history, and local damage to the implant shell is expected during the peeling process in the subsequent stripping process. In addition, during the process of salt removal method and foam engraving method for making a concave-convex shape using salt, brushing, and foam materials, a significant amount of residues (debris) of the silicon material are present in the implant shell due to manual work by hand.

The difference in the degree of cross-linking due to the non-uniformity of the coating thickness described above greatly affects the strength and free volume characteristics, and acts as a key cause of bleeding and blooming. In addition, the presence of excessive dents in the shell of the implant causes the implant to rupture as the thickness becomes thin.

Among the problems of the manufacturing process of the conventional silicone breast implant, the non-uniformity of the coating thickness can be clearly identified in a number of documents reported in academia. As shown in FIG. 6, it was confirmed that the physical property deviations of tensile strength, elastic modulus, elongation rate and tear strength were very severe, and it was found that the problem in the conventional manufacturing process was more serious than expected. In general, if the coating thickness of the implant is constant, the thermal history is transmitted evenly and the degree of cross-linking becomes uniform, so that the mechanical properties appear uniformly. However, when the coating thickness is non-uniform, a variation in thermal history occurs and a difference in the degree of cross-linking occurs, resulting in severe variation in mechanical properties. These deviations are directly related to the causes of bleeding and blooming and cause implant rupture.

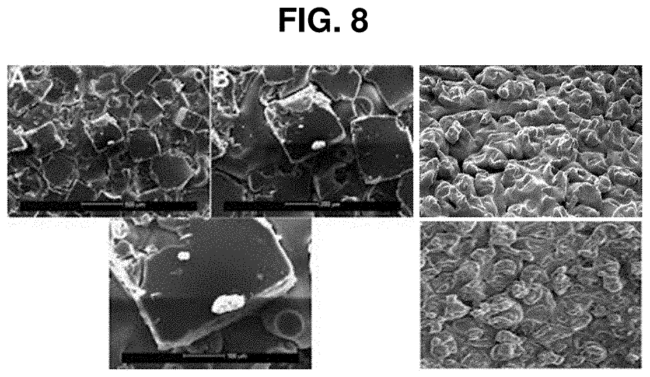

In addition, the presence of debris present on the implant surface was also confirmed through FIG. 8. FIG. 8 is a SEM photograph of the surface of the implant manufactured by salt removal method and foam engraving method. Depending on the degree of adhesion to the surface, the debris may exist for a long time inside the human body and come off through friction with scar tissue or capsular contracture tissue, which may act as a source of anaplastic large cell lymphoma. In particular, if the size of the debris is very small, it may act as a cause of breast implant illness symptoms.

Symptoms of anaplastic large cell lymphoma are said to develop on average between 7 years and 10 years after breast implant surgery, and the problem was mainly raised in patients who had a concave-convex type breast implant, and pain or swelling due to the formation of body fluids on the implant surface is observed. In some patients, a lump is felt around the surface of the breast implant or in the lymph nodes in the armpit, and in rare cases, the lymph nodes become enlarged, skin rash, fever, weight loss, and the like are accompanied.

Prior to the filing of the present invention, there is no clear explanation of the path by which anaplastic large cell lymphoma was developed around the concave-convex breast implants. According to theoretical estimation, the possibility of biofilm formation and genetic factors are considered as the main causes.

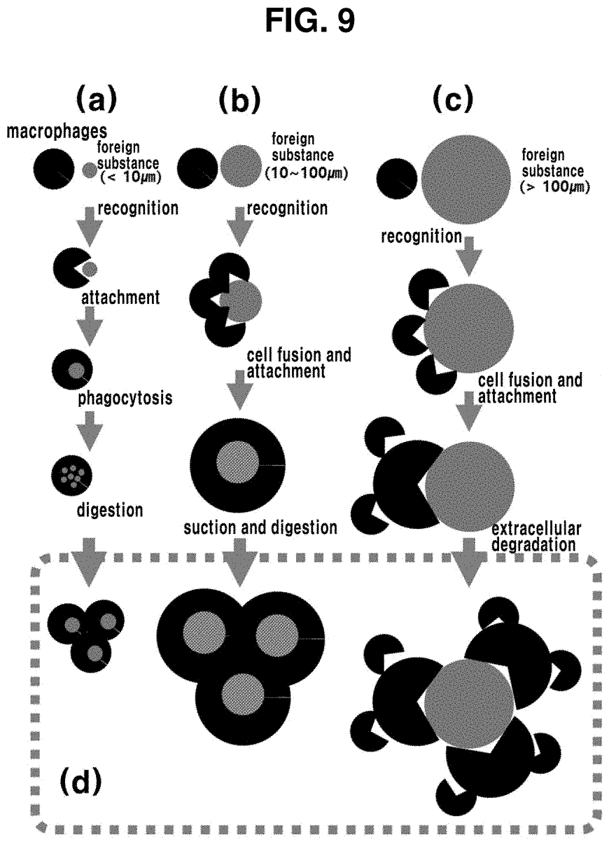

After diligent efforts to identify the cause, the present inventors found that the immune response of the human body is complexly linked to the debris present separated from the surface of the implant including organic matter that leaked out in a large amount due to implant rupture or bleed through a cross-linking structure with an insufficient degree of cross-linking. The general immune response of the human body works differently depending on the size of the foreign substance. Referring to FIG. 9, macrophages, which play a key role in the immune response of the human body, have different phagocytosis according to the size of foreign substances. Usually, when the foreign substance is less than 10 μm, it is absorbed and digested through recognition, attachment, and phagocytosis alone, and then discharged out of the body through metabolism. When the size of the foreign material is between 10 μm and 100 μm, several macrophages cooperate for phagocytosis. And when the size of the foreign material is 100 μm or more, phagocytosis usually proceeds by cell fusion in which several macrophages are converted into single giant macrophage and the number of macrophages participating in cell fusion may increase depending on the size of the foreign material. The above case is effectively applied to foreign substances that are decomposed by macrophages. However, for foreign substances that cannot be decomposed, such as silicone resin, macrophages mobilize various enzymes present in the human body to decompose them or take measures to adjust the pH, and, if necessary, mobilize active oxygen to decompose the foreign substances. Such phagocytosis does not cause a major problem to the human body in the case of biodegradable foreign substances. However, when macrophage phagocytosis is activated against a silicone material that is not biodegradable, secreted active oxygen (retaining radicals) and acidification according to pH control have a fatal effect on collagen fibers as well as silicone materials.

In addition, the points are that the silicone resin has a relatively stable molecular structure compared to collagen fibers, and if the highly reactive cross-linking agent included in the cross-linking of the silicone resin not being participated in the reaction is introduced into the human body due to the various causes mentioned above, it can have fatal effects on the human body. In other words, the danger of silicone-based cross-linking agents is that they have the ability to form a complex of elastomers that are not decomposed as they are participating in cross-linking between collagen fibers flocked together with macrophages to decompose silicone organic materials including them. Active oxygen (retaining radicals) secreted at this time and acidification due to pH control affect various reactive sites of collagen fibers to change them into a chemically unstable structure and act in a way that cross-linking with silicone-based cross-linking agents proceeds smoothly. When such a complex or silicone-based cross-linking agent moves through blood vessels alone, it can cause various symptoms in the human body. In particular, considering the characteristics of implants after the 6th generation, the fact is that a fairly large amount of cross-linking agent is used and the fact is that the cross-linking agent that did not participate in the reaction is highly likely to bleed into the human body because the degree of cross-linking of the silicone resin is not higher than expected.

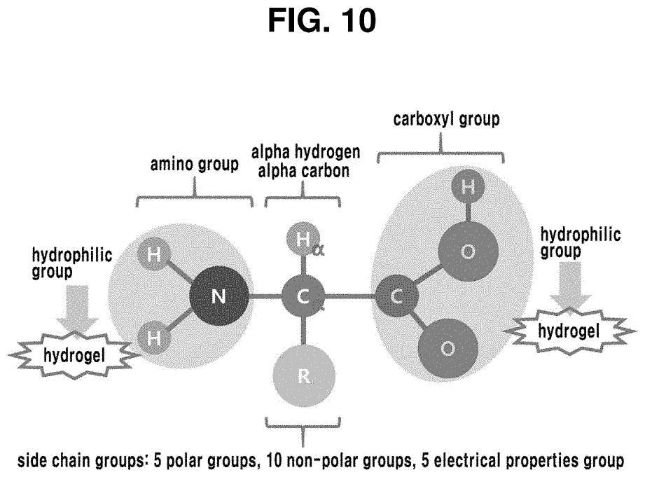

FIG. 10 is a brief description of the structural specificity of amino acids, which are components of collagen fibers. As shown in the figure, amino acids have various reaction sites, and in particular, alpha oxygen and alpha hydrogen are easily converted into radicals by acid catalysts and active oxygen, so that they can easily form chemical bonds with silicone resins. Although this chemical bond is initially an elastomer structure, it is also possible to convert into a rigid cross-linked structure as a considerable time elapses by the action of active oxygen continuously generated inside the human body. In addition, the regular existence of amino groups and carboxy groups does not cause a problem in the state of collagen fibers alone, but in the presence of chemical bonds between collagen fibers and silicone resin complexes, they are converted into hydrogels and become fatal substances to the human body. Hydrogel is a hydrophilic (polar) cross-linked polymer that can contain a large amount of water, and is not decomposed and accumulated while traveling all around the body in the form of a sticky mass, which can cause problems.

Looking at a representative case of anaplastic large-cell lymphoma briefly, there is a lymph node as an organ that plays an important role in the human immune system. The lymph node is a kidney bean-shaped organ filled with lymphocytes that are involved in immune function as a type of agranular white blood cell. Lymph nodes are present in all regions of the human body, typically in the clavicle, armpit, abdomen, groin (between the two legs) and popliteal (the concave part of the knee bent). Their function is like a sieve that filters body fluids, and when various foreign substances, including organic matter such as silicone resin, that have come through lymphatic vessels from other human tissues, enter the lymph nodes, lymphocytes and macrophages act by the body's immune response. In addition, when the above-mentioned hydrogel formed by chemical cross-linking of a complex with a silicone organic material, collagen fibers, and a silicone-based cross-linking agent exists, it will absorb surrounding moisture and expand. These symptoms can be observed with the naked eye as lymph node swelling.

As described above, the silicone-based cross-linking agent will provide a cause of chemical cross-linking between collagen fibers mobilized by immune responses in the human body, various active oxygen and silicone organic materials, and as time elapses, the strength of the cross-linked structure can become stronger, so that it may serve as a key factor contributing to the incidence of various types of anaplastic large cell lymphoma.

On the other hand, the introduction of a macro or micro-texture (concave-convex) shape to the surface of a silicon breast implant was aimed at preventing capsular contracture with the function of allowing the implant to be placed in place after surgery. However, the latter function was effective, but it is reported in clinical science and academia that the prevention of the former was not at all effective.

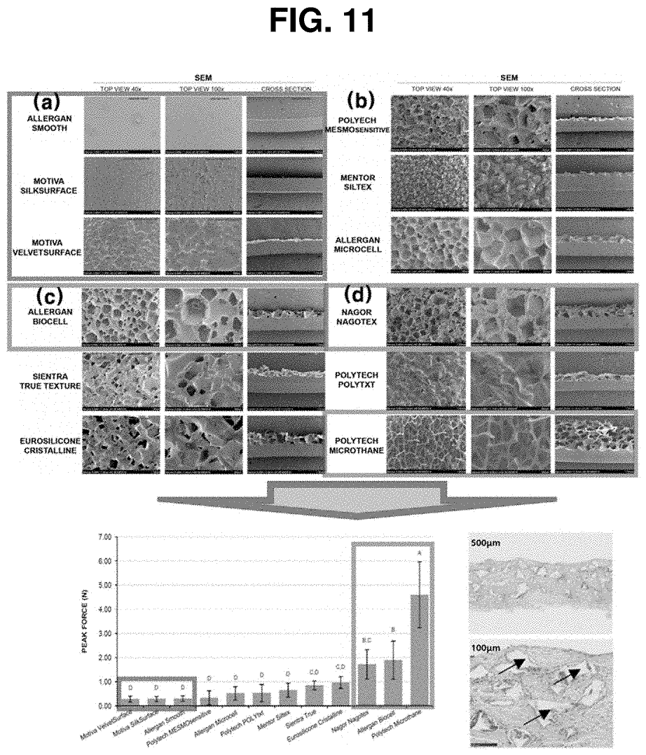

FIG. 11 is a result of SEM measurement and adhesion strength measurement for macro- and micro-texture (concave-convex) characteristics of the surface of a silicon implant. As for an adhesive strength, various types of macro and micro-textured types were implanted in experimental mice, and after 6 weeks had elapsed, the force required to detach the grafted specimens from the tissue surface of the mice was measured. As shown in the figure, it can be seen that the adhesive strength of the specimen marked with a red square is significantly higher than that of the specimen marked with a blue square. In addition, it was clearly confirmed from the 100 μm SEM measurement result that the capsular contracture was formed in the Polytech Microtan product with the highest adhesive strength. Based on these results, academia reported that an implant with a high degree of convex-concave surface acted as the main cause of capsular contracture formation. Therefore, based on these results, the industry has released a macro-texture-based new product that breaks away from micro-texture, and the decreasing trend of capsular contracture needs to be confirmed in the future.

The present inventors have confirmed that the silicone breast implant provided in FIG. 11 is a product of the 7th to 8th generations having a hard shell (outer layer) of several layers filled with cohesive gel. It should be noted that the characteristic of these products is that the outer layer is hard and thick, as also mentioned in the background art of the invention. In particular, the fact that the outer layer has become hard means that the content of the cross-linking agent is significant in the composition of the silicone resin.

Silicone resins are generally excellent in cold resistance, and maintain their inherent soft properties even at −60 to −70° C. However, as the degree of cross-linking increases as the amount of cross-linking agent increases, the temperature exhibiting soft properties rises to room temperature. This fact provides an important clue for another interpretation of FIG. 11. That is, it has been newly revealed that the cause of capsular contracture formation in experimental mice is the hardness of the surface rather than the concave-convex shape of the implant surface itself.

If the surface of the implant is concave and convex and hard, especially if it maintains a sharp shape, the surface of the implant acts similar to abrasive paper (sandpaper) in the process of continuous friction with the living tissue, which is a soft elastomer. Therefore, the living tissue is damaged, and scar tissue can be strongly formed by chronic inflammation and the immune response of the living body. It was confirmed that if the silicon material remains on the implant surface in a series of processes, it leads to the formation of capsular contracture as described above.

The present inventors have identified through careful analysis and reinterpretation of various academic data that the cause of capsular contracture formation, rupture, and incidence of anaplastic large cell lymphoma is directly related to bleeding and bleeding of implant contents, and to solve this problem, we developed an integrated implant in the aspects of structure and composition.

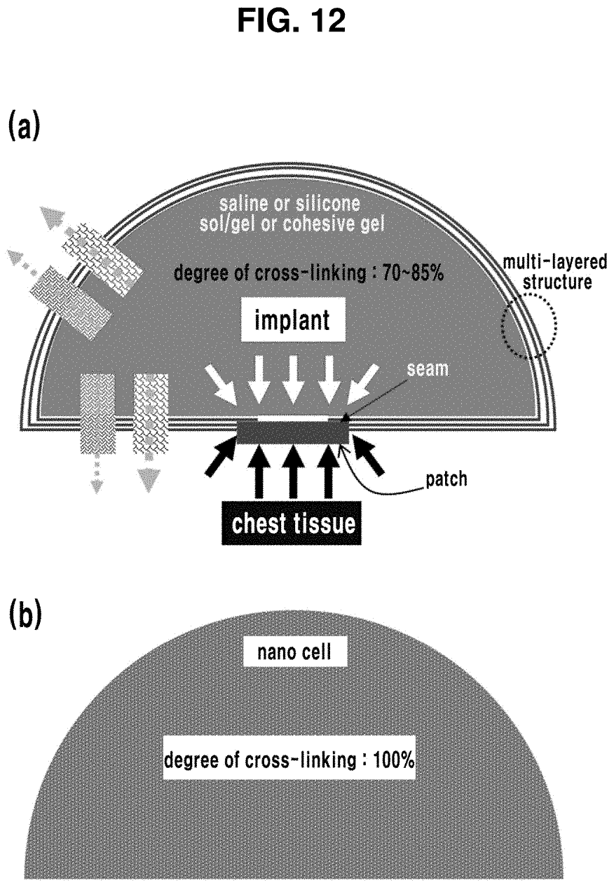

Referring to FIG. 12, the structural and compositionally integrated implant as (b) presented in the present invention provides a structurally integrated implant in which no seams, patches, and multi-layer structures exist in order to prevent bleeding and blooming caused by long-term concentration of stress and load between implant and breast tissue around the seam and multi-layer structure region, which is a structural defect of the existing implant (a).

In addition, an integrated implant having uniformity in compositional aspect is provided to minimize unreacted silicone resin caused by imperfection of the degree of cross-linking of the inner and outer layers of the implant, presence of unreacted silicone-based cross-linking agent, and instability of dimensional stability associated with free volume.

The present inventors have identified that the breast implant illness symptoms are caused by the impact applied by the action and reaction caused by the weight or strength of the implant and the mechanical interaction between the implant of such structure and the living body.

Breast implant illness symptoms are newly revealed symptoms in the 6th and 8th generation implants, which have a hard and thick outer layer structure composed of cohesive gel and multiple layers. Academia suspects the body's immune response to these symptoms, especially autoimmune diseases, and is looking for solutions assuming that the main causes are the reactivity of specific substances applied to breast implants and estimation by biofilm.

The present inventors have found that the above-mentioned causes are also partially involved, and clarified that the more important cause is the biomechanical interaction between the implant and the body tissue through various literature investigations and continuous research. It was also confirmed that the weight and strength of the implant also acted as a factor.

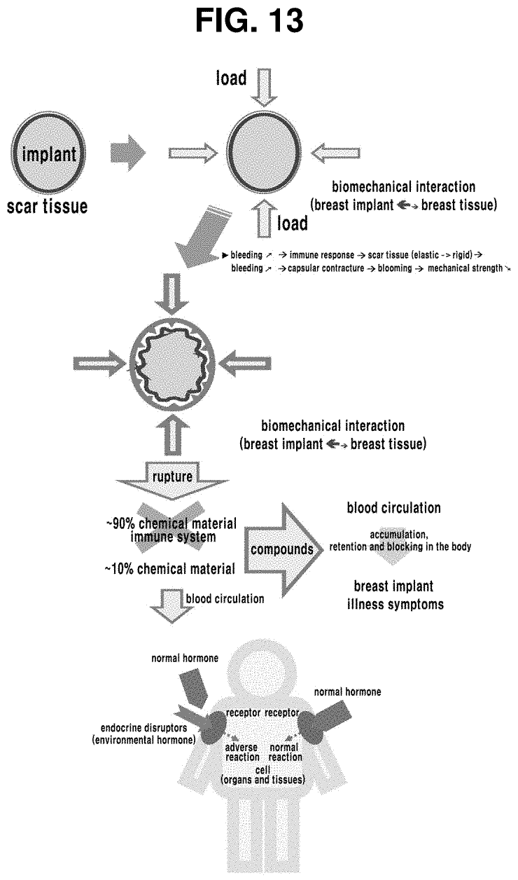

FIG. 13 schematically shows the effect of autoimmune disease and the reactivity of the silicone material on the symptoms of breast implant illness. The scar tissue formed around the breast implant is formed through a natural healing process as part of the body's immune response. This scar tissue is composed of collagen fibers of the elastomer. However, when the implant contents are gradually bleeding out by the load applied by the mechanical interaction between the implant and the breast tissue, the scar tissue becomes hard through an immune response. As the strength of the load applied through the breast tissue becomes stronger due to the biomechanical interaction of the hardened scar tissue, and the amount of bleeding of organic matter increases, a capsular contracture is formed through a series of immune reactions. Also, when the implant receives a stronger load from the capsular contracture tissue, blooming occurs, eventually weakening the mechanical strength of the implant's outer layer, leading to rupture.

When a large amount of silicon organic material flows into the human body due to the rupture, a significant portion thereof forms the complex described above by the human immune system, and some components affect each tissue of the human body through blood circulation. In particular, silicone-based cross-linking agents act similar to endocrine disruptors (environmental hormones) and cause abnormal reactions in organs and tissues in the body. Through this series of processes, normal hormones in the human body are not able to function properly, providing the cause of autoimmune diseases including various side effects. And in the case of the above-mentioned complex, it causes accumulation, retention, and blocking in organs and tissues including blood vessels in the body through blood circulation, causing breast implant illness symptoms of unknown cause.

Breast implant illness symptoms can be found at some part in the autoimmune disease mentioned above and the reactivity of the silicone material. However, it is insufficient to explain the numerous symptoms, and there are a lot of unexplained parts, so after additional research, the decisive cause was discovered.

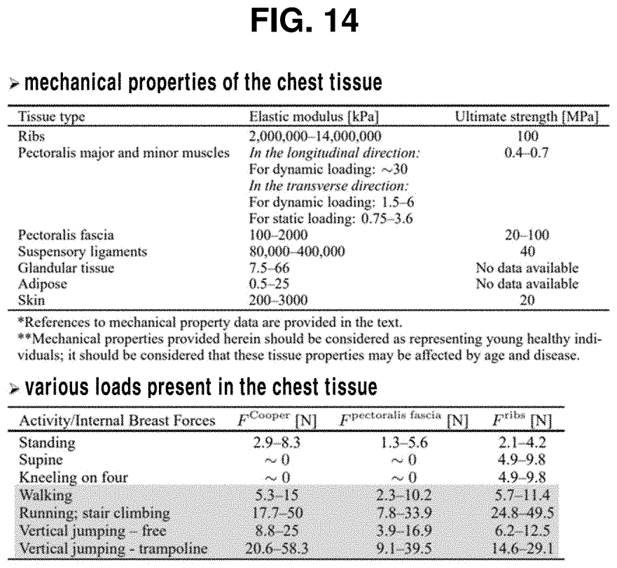

FIG. 14 is an excerpt from some of the research results of the prior art on the measurement of mechanical properties of various tissues existing in the chest region of the human body and the loads in the chest tissue caused by various human activities. The chest region of the human body is made up of ribs, muscles, and numerous ligaments, and as a result of measuring the load applied to the chest region when these chest tissues are in a standing position, kneeling prone position, walking, running, jumping, and lying on the back, which are human physical activities, it was confirmed that considerable force was applied when walking, running, or jumping vertically. In particular, when there is intense physical activity due to numerous ligaments with very good elasticity in the chest region, the repulsive force of the ligaments is added to the weight of the implant, and the effect on the human body is judged to be beyond imagination.

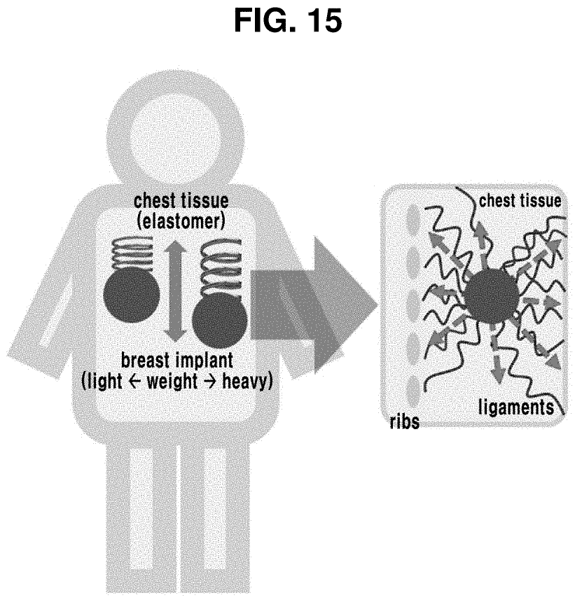

FIG. 15 is a description of the biomechanical interaction between the breast implant and the breast region tissue. The weight of breast implant is important because, when a heavy implant is placed between the typical ligaments and muscles of the chest tissue, a tremendous load is applied to the ribs, various organs, and blood vessels around the chest tissue due to the action and reaction of the body's physical activity. Also, if this load is applied continuously rather than a temporary phenomenon, the symptoms felt in each region of the body will be very large.

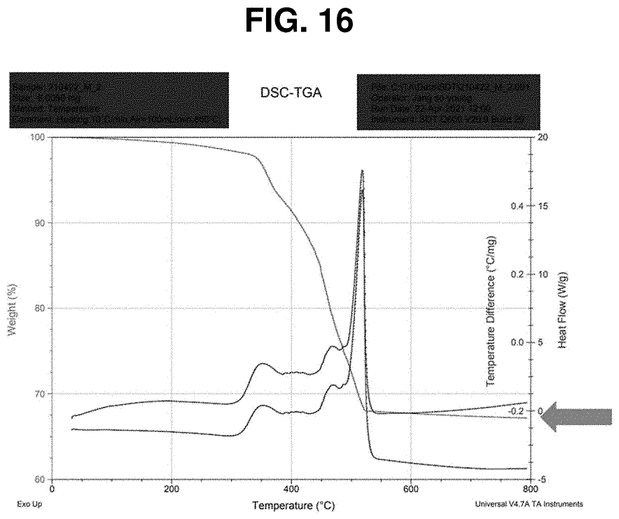

The weight and strength of the implant were small at the beginning, but the momentum for the rapid increase in weight was provided since the free volume area formed by cross-linking of the silicone resin was wide and weak, the bleeding phenomenon became severe, attempts were tried to fill the free volume with silica material chemically bonded to silicone resin to minimize the bleeding of low molecular weight silicon which is one of the methods applied from the 3rd generation implant to the present. It can be seen that the silica content used in the silicone resin currently used for breast implants is a significant level of about 60% or more. FIG. 16 is a TGA analysis result for the weight loss of the silicone resin, and the content of silica is approximately 67% as a representative silicone resin currently used in the industry.

The surface strength began to increase with the application of several thick and hard outer layers in the 6th to 8th generations, where implants filled with cohesive gel started to be applied. Surface strength showed an indirect correlation with breast implant illness symptoms, and it was confirmed that it had a direct correlation with capsular contracture formation.

The present inventor has identified through years of practical experience and continuous research that the cause of incidence of breast implant illness symptom is directly related to the biomechanical interaction between the implant and the breast tissue and the weight of the implant, and we developed an implant of radial cell structure was developed for the purpose of solving this problem.

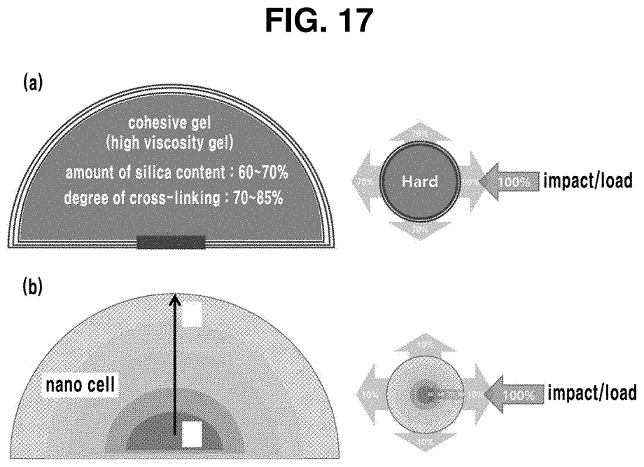

FIG. 17(a) is a general structure shown from the existing 6th generation to the current implant, and the implant filled with a high content of silica and a high viscosity cohesive gel comprises the mainstream. Because these implants are generally hard and heavy, impacts or loads resulting from biomechanical interactions are applied directly to the breast tissue (ribs, muscles, various blood vessels, and the like) causing various types of breast implant illness symptoms.

In order to innovatively solve the disadvantages of the implant, the present inventors developed an implant of a radial cell structure that controls the morphology in the geometrical aspect of various cells to dramatically lower the weight of the implant, and at the same time, perfectly absorbs or removes the impact or load applied due to biomechanical interaction.

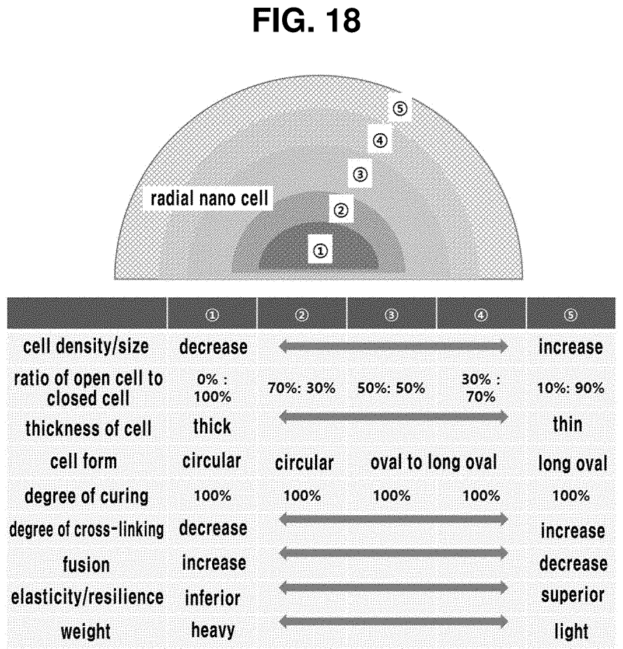

In addition, as shown in FIG. 18, the radial cell structure maximizes the elastic restoring force of the implant by arranging cells with various characteristics sequentially in all directions based on the cell in region \textcircled{1} below the center of the implant, thereby dramatically improving dimensional stability.

The cells in region \textcircled{1} below the center of the implant have a 100% circular closed cell structure, with thick cell walls and low cell density, cell size and cross-linking density, and it is a geometric structure with strong viscous properties that absorbs the impact applied to the implant from the outside of the body and the breast tissue and removes it completely. A closed cell is a micro-sized circular pore formed inside the cell in region \textcircled{1}, and is a pore formed during gas expansion in the mold.

On the other hand, in a direction from the center of the implant to the outer circumferential surface of the implant, the cell has a circular, oval, and long elliptical cell shape as shown in FIG. 18, the thickness of the cell wall becomes thinner as it travels toward the outer circumferential surface of the implant, but the cross-linking density, cell density and cell size increase, resulting in a geometric structure with strong elastic properties.

Such structure acts as a buffer by gradually absorbing the impact or load applied to the implant from the outside of the body and the breast tissue so as to be completely removed from the center of the implant. The open cell has a dumbbell-shaped ( [symbol] ) structure as micropores formed during gas expansion in the mold.

In addition, region \textcircled{1} below the center of the implant has low nanocell density, size, and cross-linking density so that the weight is relatively heavy compared to the implant in the outer circumferential direction, thereby providing the function of preventing the implant from dislodging and placing it in its normal position after implant surgery, and nanocell density, size, and cross-linking density increase as they travel toward region \textcircled{5}, which is the outer circumferential direction of the implant so that the weight is relatively light compared to the region below the center of the implant to increase the elastic restoring force, so that the effect of minimizing wrinkles, ripples, or dents of the implant after implant surgery is excellent.

The integrated radial cell structure implemented in the present invention has an excellent effect of completely absorbing and removing the load and impact applied to the breast tissue from the outside and the breast implant by effectively controlling the geometric morphology of the cell.

The technology developed through the present invention is only an example in application to the above-mentioned breast implant, and can also be applied to various implants inserted into the human body.

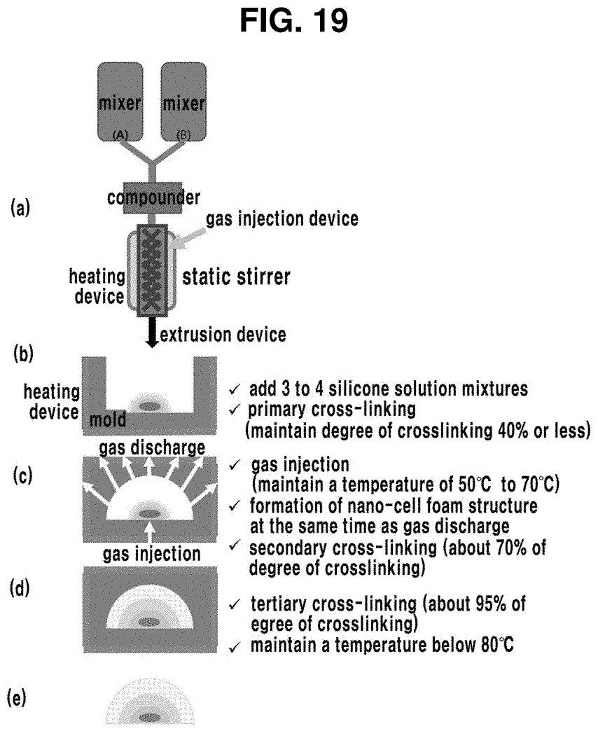

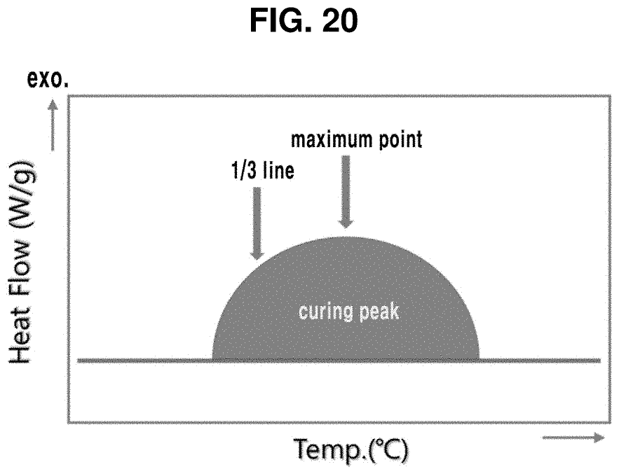

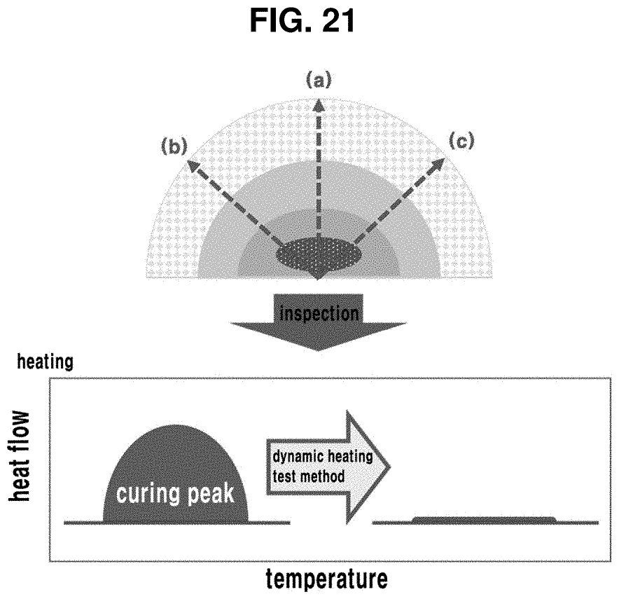

A process of manufacturing a breast implant having an integrated radial silicone-filled cell structure according to an embodiment of the present invention will be described in more detail with reference to FIG. 19.

FIG. 19 is a schematic diagram illustrating a manufacturing method of a breast implant having an integrated radial silicone-filled cell structure of the present invention according to an embodiment of the present invention.

Referring to FIG. 19, in the method of manufacturing a breast implant having an integrated radial silicone-filled cell structure of the present invention according to an embodiment of the present invention comprises the steps of: preparing a silicone solution mixture (S10); injecting the silicone solution mixture (S20); forming a plurality of radial silicon-filled cell structures (S30); and may further comprise the steps of: heating the silicon-filled cells (S40); and obtaining a molded implant for human injection (S50).

In other words, the manufacture of a breast implant with an integrated radial cell structure is performed in a way that, as shown in FIG. 19, the liquid silicone rubber mixture is prepared as a silicone solution mixture that is finely dispersed and distributed using a primary compounder and a secondary static stirrer; then the liquid silicone mixture according to the degree of cross-linking is put into the same mold as the implant shape to perform primary cross-linking; then secondary cross-linking is carried out at the same time as foaming by adding air or nitrogen or carbon dioxide gas that has been heated and sterilized to an appropriate temperature; and after completion of discharging the injected gas, the final tertiary cross-linking completes the manufacture of a breast implant having an integrated radial cell structure having the shape of a mold.

Looking at detailed steps, the step of preparing the silicone solution mixture according to the present invention (S10) is a step of preparing a plurality of silicone solution mixtures having different degrees of cross-linking.

The silicone resin used in the present invention is a two-component type liquid silicone rubber (LSR), in which the mixture A in which the main resin and the catalyst are mixed, a cross-linking agent, an auxiliary resin, and, if necessary, a filler (silica) are mixed It consists of mixture B. The silicone solution injected into the mold is preferably a uniformly dispersed and distributed mixture obtained by mixing the mixture A and the mixture B in equal proportions and passing through a static mixer.

Mixtures A and B of silicone solution are very sticky cream-like liquids with a viscosity of 100,000 cps (centi poise), respectively, and it is preferred to use a static mixer because a mixture having uniform dispersion and distribution characteristics cannot be obtained with a general stirrer. The state of poor dispersion and distribution in which the various components contained in the mixture are not uniformly mixed results in a sharp decrease in mechanical strength, which is the cause of rupture, due to the non-uniform characteristics of the degree of cross-linking and free volume.

The mixing ratio of mixtures A and B of the silicone solution is preferable to select under the condition wherein the temperature of the peak width is between 5° C. and 30° C. based on the maximum temperature of the hardening peak obtained through the thermal analyzer, it is more preferable to select under the condition wherein the temperature of the peak width is between 5° C. and 20° C. based on the maximum temperature of the hardening peak, and it is most preferable to select the ratio of the silicone solution mixture A and B under the condition wherein the temperature of the curing peak width is between 5° C. and 10° C.

The width of the hardening peak is an important factor in determining the ease of cross-linking and the characteristics of free volume, and a wide curing peak means that the cross-linking reaction of liquid silicone rubber proceeds in a wide temperature range, which is commonly observed in thermosetting resins with high molecular weight. In this case, the cross-linking initial reaction proceeds rapidly at a relatively low temperature, resulting in a high degree of cross-linking and a sharp increase in molecular weight, and the final stage reaction may be difficult to proceed with a final degree of cross-linking of 80% or more since the reaction proceeds at a high temperature due to the rapid decrease in reactivity between the rapidly increased high molecular weight silicones. In addition, the free volume of the cross-linked structure is generally wide. Therefore, it is important to adjust the mixing ratio of A and B of the silicone solution mixture to select a condition in which the width of the curing peak is around 10° C.

The silicone solution mixture preferably contains a silica content between 0.1 vol % and 40 vol %, more preferably contains between 0.1 vol % and 30 vol %, and most preferably may contain between 0.1 vol % and 20 vol %.

The present invention can minimize the symptoms after breast implant by reducing the weight of the cosmetic implant by reducing the silica content. To this end, the weight and shape of the implant, as shown in FIG. 18, and the silica content can be reduced through the formation of elastomer cell structure. The cell of the present invention has a variety of shapes from a round shape to an elongated oval shape, and has a different size and shape. Due to these characteristics, the weight of the breast implant of the present invention is remarkably reduced, thereby minimizing the influence of an external impact (load).

Referring to FIG. 18, the position 1 is a viscous region in which a round closed cell exists, and serves to remove an external impact (load) and serves as a center of gravity for a an implant. As it travels from 1 to 5 in FIG. 18, the size of the cell increases and the number of oval and long elliptical shapes increases, resulting in excellent elasticity (resilience). In addition, inside the cell is a hollow space, and as such a structure increases, the amount of silicone resin used can be reduced, and accordingly, the amount of silica can also be reduced. For example, the conventional amount of silica used is 60 vol % compared to the amount of silicone resin based on the existing size of 600 mL, whereas in the present invention, as the amount of silicone resin used decreases, the amount of silica used in the present invention having a plurality of cell layer structures can be reduced to at least 40 vol % based on 600 mL.

The plurality of cell structures of the present invention are in a state in which a small amount of gas is included in a mixture in which a silicone resin and a cross-linking agent are mixed, as shown in FIG. 19, and are laminated in a form containing 3 to 4 kinds of gases with different degrees of cross-linking, as shown in (b). The reason for varying the degree of cross-linking is to control the gas contents in each mixture, and the higher the degree of cross-linking, the greater the gas content. That is, in the case of the lamination of (b), the mixture at the bottom has a low cross-linking degree, so the gas content is low, and the mixture placed above has relatively high gas content. When the lamination of the silicone mixture is completed, the upper layer of the mold is covered, additional gas is injected with pressure and temperature applied, and the injected gas is discharged after a certain period of time, then a small amount of the silicone mixture as shown in (c) is swelled up as shown in (d) and filled in the mold shape, and cells with various shapes and sizes are formed according to the characteristics of the laminated silicone mixture. Therefore, if the existing breast implant was made using 600 g of silicone resin, the present invention can manufacture the same size as the existing breast implant with only 400 g of silicone resin, and accordingly, the amount of silica used in the conventional case is also 60 vol % compared to the amount of silicone resin used, whereas in the present invention, as the amount of the silicone resin is reduced, the amount of silica can be reduced to at least 40 vol % or less.

The silica content is the same in all four types of mixtures input from step (a) to step (b) of FIG. 19 in terms of w %. However, in terms of vol %, the content of silica varies according to the formation of a cell structure according to the injection and discharge of the heated gas in steps (c) and (d). In other words, as the geometric morphology of the cell is changed, the vol % of silica in the region below the center of the implant is high as shown in the FIG. MA, and the vol % of silica decreases rapidly as it getting closer to the outer circumferential surface of the implant. Due to these characteristics, the elastic restoring force of the implant was maximized and the biomechanical interaction with the breast tissue was minimized.

The silicone solution mixture is preferable that its main silicone resins and the auxiliary silicone resin except silica have a reactive group that participates in the cross-linking reaction with the cross-linking agent or when a reactor group is not provided, a molecular weight of 100,000 or more is preferable. In addition, it is preferable to manage the content of low-boiling-point organic matter between 1 wt % and 3 wt %. The presence of large amounts of low-boiling-point organic matter has a fatal adverse effect on the formation of the radial cell structure of the present invention. The content of a low-boiling-point organic matter is confirmed through TGA analysis, a thermal analyzer, and when the above weight loss is confirmed between 150° C. and 200° C. in TGA analysis, it is preferable to use it after removal using a Soxhlet extractor.

Silicone resin may be used as the main resin and auxiliary resin used in the silicone solution mixture, and the type including a rigid benzene structure in the main chain and side chains of the resin is excluded. In addition, a type manufactured by an addition reaction method using a platinum catalyst is preferable.

In the manufacture of the implant in the present invention, the silicone solution is mixed with the mixture A and the mixture B in equal proportions and passed through a static mixer to use a mixture with uniform dispersion and distribution.

The static mixer used to prepare the silicone solution mixture is equipped with a heating device to ensure uniform dispersion and distribution of the mixture and a certain level of degree of cross-linking, and a gas injection device can be installed at an appropriate location for the formation of a cell structure.

The step of introducing the silicone solution mixture (S20) is a step of sequentially introducing the plurality of silicone solution mixtures into the mold.

The homogeneous silicone solution mixture controls the speed of passing through a static mixer with a heating device, so that four types of mixtures with different degrees of cross-linking are sequentially and quickly introduced into the mold with a heating device [FIGS. 19(a) and (b)]. The degree of cross-linking depends on the residence time inside the static mixer and the temperature conditions applied through the heating device.

In the above process, the degree of cross-linking of the mixture first added to the mold is the lowest, and the degree of cross-linking increases in the order in which it is sequentially added. The reason for setting the difference in the degree of cross-linking is that it is a key factor influencing the size and shape of cells. In general, the lower the degree of cross-linking, the more spherical closed cells with thick cell walls are formed, and the higher the degree of cross-linking, the more oval open cell structures with thin long cell walls are mainly formed.

The degree of cross-linking of the four mixtures above is preferable to maintain in a way that 10% or less for the bottom mixture, 10%˜20% for the second mixture, 20%˜30% for the third mixture, and 30%˜40% for the last mixture.

In addition, the rate of injection of the silicone solution mixture and the degree of cross-linking are determined and confirmed through hardening peak analysis according to the dynamic and static heating test method using a thermal analyzer.

The step of forming a plurality of radial silicone-filled cell structures (S30) is a step of forming a plurality of radial silicone-filled cell structures by injecting a gas heated in the mold into a plurality of silicon solution mixtures having different degrees of cross-linking.

When four types of silicone solution mixture are injected into the mold, the upper portion of the mold is covered and fixed with bolts [FIG. 19(c)]. Then, a certain amount of air or nitrogen heated and sterilized to an appropriate temperature is injected through the gas inlet at the lower portion of the mold.

The gas is injected into the dead central region of the lower portion of the mold, and in particular, the central position of the first injected mixture is preferable. The first mixture acts as the centerpiece of the breast implant, and functions to put the implant in its normal position after the breast implant.

In addition, gas is discharged through more than 17˜25 outlets installed on the upper portion and side surfaces of the mold, and specifically, it is preferable to install one outlet at the very center of the upper portion of the mold, and then install eight in a circle, and install 8˜16 on the side surfaces of the upper portion of the mold. If there are more than 25 gas outlets, a radial cell structure having the characteristics shown in FIG. 18 is formed, but the elongated oval-shaped cell, which is the core of elastic restoring force, is not perfectly formed and thus does not perform its functions. In addition, the outlets quickly discharge the injected gas simultaneously, and are closed quickly. Otherwise, the proportion of open cells becomes excessively high, resulting in a decrease in elastic restoring force.

According to the injection rate, content, discharge rate, and opening/closing speed of the heated air or nitrogen or carbon dioxide gas, the silicone solution mixture expands according to the shape of the mold to form a cell structure having various sizes and shapes [FIG. 19(d)]. In addition, the flow of heat is facilitated through the cell structure formed according to the gas injection, so that secondary cross-linking proceeds.

The size and shape of the cell directly depend on the silicon solution mixture injection rate, the degree of cross-linking, and the content of air or nitrogen or carbon dioxide gas injected in the step (c), which are the conditions of steps (a) and (b). The content amount of the injected gas is expressed as a function of the temperature applied to the gas.

The temperature of the gas injected into the mold is preferably between 90° C. and 100° C. Specifically, it is more preferably between 50° C. and 70° C. In more detail, the maximum temperature of the curing peak obtained through thermal analysis is most preferable. At this time, the gas injection time and speed is preferable to select in a region where the degree of cross-linking is 70% or less. When the degree of cross-linking is 70% or more, it should be noted that low-boiling-point organic substances and gases that are not completely discharged from the tertiary cross-linking in step (d) may be trapped in the implant shell region and large cells may be formed. The cross-linking degree of 70% is confirmed through curing peak analysis according to the dynamic and static heating test method using a thermal analyzer.

In addition, the cell structure of the present invention is formed according to a gas injection method, and the gas injection may be sequentially applied in either step or both steps of step (a) or step (c) of FIG. 19. A preferred gas injection method is to be applied sequentially in both steps, wherein step (a) acts as a nucleating agent for cell formation, and step (c) enables cells with various shapes and characteristics from the lower-center region of the implant toward the implant shell region are effectively formed based on the cells formed in step (a).

In the present invention, the use of the chemical foaming method using organic and inorganic chemical foaming agents and the physical foaming method using a low-boiling-point organic material or solvent are banned since various types of residues that inevitably remain in the foam after the formation of the foam structure may cause the problem of biocompatibility with the human body.

The heating of the silicon-filled cells (S40) is a step of heating the plurality of radial silicon-filled cells to a temperature range of 70° C.˜100° C. at the same time as discharging the injected gas.

After the gas injection and discharge are completed, it is preferable to proceed with the tertiary cross-linking between 90° C. and 100° C. In detail, it is more preferable to proceed between 70° C. and 80° C. In more detail, it is most preferable to select the temperature of the left 1/3 line based on the maximum temperature of the curing peak obtained through the thermal analyzer. In addition, as the most desirable temperature condition, it is strongly recommended to select the temperature of the 1/4 line or 1/5 line on the left side based on the maximum curing peak temperature as the size of the implant increases and the weight becomes heavier.

In case, if the temperature of the right 1/3 line is selected, the degree of surface cross-linking of the implant rises sharply, resulting in an effect of blocking the heat flow to the inside, so the degree of cross-linking inside does not increase in proportion to the degree of cross-linking of the surface, and therefore, ultimately, the degree of cross-linking inside the implant cannot reach 90% or more. And when the difference in the degree of cross-linking between the surface and the inside becomes between 30% and 40%, a trace amount of low-boiling-point organic substances that may be generated by the progress of the internal cross-linking reaction cannot escape through the dense cross-linking structure of the surface, thereby forming large cells between the interfaces.

The selection of the time required for the tertiary cross-linking is also confirmed through the analysis of the curing peak according to the dynamic and static heating test method using a thermal analyzer.

The step of obtaining a molded implant for human injection (S50) is a step of cooling the heated plurality of silicon-filled cells to room temperature at a constant rate to obtain a human body implant.

Finally, when the tertiary cross-linking is completed, the temperature of the mold is cooled to room temperature at a constant rate, and then the upper and lower portions of the mold are separated to obtain a breast implant with an integrated radial cell structure of the present invention [FIG. 19(e)].

The implant having the integrated radial cell structure obtained through the above manufacturing process is completed through various verification procedures.

First, the degree of cross-linking is closely related to the bleeding and blooming phenomena, and as the degree of cross-linking approaches 100%, the bleeding and blooming phenomena are minimized. As shown in the drawings below, the degree of cross-linking is confirmed through curing peak analysis according to the dynamic heating test method using a thermal analyzer by taking a certain amount of samples to check the degree of cross-linking for each region of the implant obtained above.

A degree of cross-linking of 100% can be confirmed that the curing peak measured according to the dynamic heating test procedure is not completely visible as shown in the drawing.

As a result of verification of the degree of cross-linking for the implant having the integrated radial cell structure obtained above, when the degree of cross-linking is 95% or less, it means that there is a small amount of unreacted material that did not participate in the cross-linking reaction of the silicone resin and the cross-linking agent. The unreacted material acts as a factor in the occurrence of bleeding and blooming from the implant to the breast tissue. This is also greatly influenced by the characteristics of the free volume according to the degree of cross-linking.

The possibility of bleeding and blooming is additionally checked using a Soxhlet extraction device as a final verification operation for an implant having an integrated radial cell structure that has obtained 100% cross-linking and verified through the above thermal analyzer. The solvents used for Soxhlet extraction are 3 to 4 types of polar solvents such as alcohol including water, and the temperature condition is selected to be 10° C. lower than the boiling point of each solvent used.

Meanwhile, the above detailed description should not be construed as limiting in all aspects, but should be considered as exemplary. The scope of the present invention should be determined by a reasonable interpretation of the appended claims, and all modifications within the equivalent scope of the present invention are included in the scope of the present invention.

What is claimed is:

1. In a human body implant,
an integrated radial silicone-filled cell-structured human body implant comprising:
a first silicone-filled cell including a silicone filling material, in which the silicone filling material is formed in the center of the implant; and
a second silicone-filled cell surrounding an outer surface of the first silicone-filled cell, being formed radially around the center of the implant, and including a silicone filling material formed with a cross-linking density different from a silicone cross-linking density of the first silicone-filled cell.

2. The integrated radial silicone-filled cell-structured human body implant according to claim 1, characterized in that the human body implant is used for at least one selected from a group consisting of chest, breast, buttocks, nose, chin, forehead, calf, thigh, and wrinkles.

3. The integrated radial silicone-filled cell-structured human body implant according to claim 2, characterized in that the human body implant is used for chest or breast,
wherein the human body implant has a semi-circular shape structure,
wherein the first silicon-filled cell is formed in a semi-circular shape in the center of a lower surface of the implant of the semi-circular shape, and
wherein the second silicon-filled cell surrounds an outer surface of the first silicon-filled cell in a semicircular shape and is radially formed around the center of the implant.

4. The integrated radial silicone-filled cell-structured human body implant according to anyone of claim 1, characterized in that the degree of cross-linking of the human body implant is 95% or more.

5. The integrated radial silicone-filled cell-structured human body implant according to anyone of claim 1, characterized in that the silicone filling material includes a silicone resin, a cross-linking agent, a catalyst, and a silica,
wherein the total content of silica contained in the implant is between 0.1 vol % and 40.0 vol % in a temperature range between −20° C. and 40° C. based on the volume of the implant.

6. The integrated radial silicone-filled cell-structured human body implant according to anyone of claim 1, characterized by comprising a plurality of silicone-filled cells disposed sequentially on an outer surface of the second silicone-filled cell.

7. The integrated radial silicone-filled cell-structured human body implant according to anyone of claim 6, characterized in that the first silicon-filled cell has a cell density of 100% of a circular closed cell and a small-sized geometric cell structure,
wherein the second silicon-filled cell has an open cell ratio close to an ellipse of a cell density between 30% and 70% and a geometrical cell structure whose size is relatively larger than that of the central region, and
wherein the silicon-filled cells formed on the outermost surfaces of the plurality of silicon-filled cells has a cell density of about 90% of a long elliptical closed cell and a geometrical cell structure having the largest size.

8. In a manufacturing method of a human body implant,
a manufacturing method of an integrated radial silicone-filled cell-structured human body implant characterized by comprising the steps of:
preparing a plurality of silicone solution mixtures having different degrees of cross-linking (step S10);
sequentially introducing the plurality of silicon solution mixtures into a mold (step S20); and
forming a plurality of radial silicon-filled cell structures by introducing a heated gas into a mixture of a plurality of silicone solutions having the different degrees of cross-linking (step S30).

9. The manufacturing method of an integrated radial silicone-filled cell-structured human body implant according to claim 8 characterized by further comprising the steps of:
heating the plurality of radial silicon-filled cells to a temperature range of 70° C. to 100° C. at the same time as discharging the introduced input gas (step S40); and
obtaining a human body implant by cooling the heated plurality of silicon-filled cells to room temperature at a constant rate (step S50).

10. The manufacturing method of an integrated radial silicone-filled cell-structured human body implant according to claim 8, characterized in that in the step S10, the plurality of silicone solution mixtures having different degrees of cross-linking has a degree of cross-linking of 40% or less,
characterized in that in the step S30, a plurality of radial silicone-filled cells formed by injecting the heated gas into a plurality of silicone solution mixtures having different degrees of cross-linking has a cross-linking degree of 70% or less, and
characterized in that in the step S40, the heated plurality of silicon-filled cells has a cross-linking degree of 95% or more.

11. The manufacturing method of an integrated radial silicone-filled cell-structured human body implant according to anyone of claim 8, characterized in that the step S10 includes the step of passing the prepared mixture of the plurality of silicone solutions through a static stirrer having a heating device and a gas injection device.

12. The manufacturing method of an integrated radial silicone-filled cell-structured human body implant according to anyone of claim 8, characterized in that in the step S30, the mold has the shape of a implant corresponding to a human body to be implanted,
wherein the heated gas is the gas injected through the lower hole of the mold and being heated by a heating device installed on an outer surface of the mold.

13. The manufacturing method of an integrated radial silicone-filled cell-structured human body implant according to anyone of claim 8, characterized in that the implant is composed of an upper portion and a lower portion.

14. The manufacturing method of an integrated radial silicone-filled cell-structured human body implant according to anyone of claim 8, characterized in that the human body implant is used for at least one selected from a group consisting of chest, breast, buttocks, nose, chin, forehead, calf, thigh, and wrinkles.
